(19) United States
(12) Patent Application Publication
(10) Pub. No.: US 2006/0147396 A1
(43) Pub. Date: Jul. 6, 2006

(54) Composition containing ascorbic acid

(75) Inventor: Aldo Monello, Savigny Sur Orge (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/322,216

(22) Filed: Jan. 3, 2006

Related U.S. Application Data
(60) Provisional application No. 60/641,741, filed on Jan. 7, 2005.

(30) Foreign Application Priority Data
Jan. 3, 2005 (FR) 05 50008

(51) Int. Cl. A61K 8/81; A61K 8/73

(52) U.S. Cl. 424/59; 424/70.13; 424/70.16

(57) ABSTRACT

The invention relates to a composition in oil-in-water emulsion form containing ascorbic acid or a derivative thereof, at least one polysaccharide hydrophilic gelling agent, at least one C₁₆-C₂₂ fatty acid sorbitan ester, at least one ethoxylated fatty acid ester and at least one polymer containing a sulpho-functional monomer.

### REFERENCE TO PRIOR APPLICATIONS

This application claims priority to U.S. provisional application 60/641,741 filed Jan. 7, 2005, and to French patent application 0550008 filed Jan. 3, 2005, both incorporated herein by reference.

### FIELD OF THE INVENTION

The invention relates to an oil-in-water emulsion comprising ascorbic acid and/or a derivative thereof, a polysaccharide hydrophilic gelling agent, a sorbitan ester, an ethoxylated fatty ester and a sulpho-functional polymer.

In a preferred embodiment a composition in oil-in-water emulsion form is described comprising ascorbic acid and/or a derivative thereof, at least one polysaccharide hydrophilic gelling agent, at least one C₁₆-C₂₂ fatty acid sorbitan ester, at least one ethoxylated fatty acid ester, and at least one polymer containing a sulpho-functional monomer. In other preferred embodiments the composition exhibits good stability, in particular after 8 days at 55° C.

Also making up a part of the invention is the use of the invention compositions for the care and make-up of keratin materials.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The introduction into cosmetic compositions of various actives intended to impart specific treatments to the skin and/or hair is known. Some of these actives, however, have the drawback of being unstable in aqueous media and of readily degrading on contact with water, particularly as a result of oxidation phenomena. Hence they rapidly lose their activity over time, and this instability runs counter to the required efficacy.

One long-pursued aim, for instance, has been to formulate ascorbic acid or vitamin C, owing to its numerous beneficial properties. In particular, ascorbic acid stimulates the synthesis of connective tissue and especially of collagen, reinforces the defences of skin tissue against external attacks such as ultraviolet radiation and pollution, compensates vitamin E deficiency in the skin, depigments the skin, and possesses a free-radical scavenger function. These two latter properties make it an excellent candidate as a cosmetic or dermatological active for combating or preventing skin ageing. Unfortunately, owing to its chemical structure (alpha-keto lactone), ascorbic acid is highly sensitive to certain environmental parameters and particularly to oxidation phenomena. The result is rapid degradation of ascorbic acid formulated in the presence of these parameters, and more particularly in the presence of oxygen, light, and metal ions, depending on the temperature, or else under certain pH conditions (Pharm. Acta Helv., 1969, 44, 611-67; STP Pharma, 1985, 4, 281-6).

In particular, a cosmetic emulsion containing ascorbic acid which is degraded exhibits, generally, a substantial fall in viscosity, and phase separation; the composition therefore becomes unstable and is no longer suitable for the intended use.

### OBJECTS OF THE INVENTION

One aim of the present invention is therefore to provide an emulsion containing ascorbic acid and/or a derivative thereof that is stable, in particular for 8 days at 35° C., or even 2 months at 55° C., or even for 2 months at 45° C.

### DETAILED DESCRIPTION OF THE PREFERRED EMBODIMENTS

The inventor has discovered that such a composition may be obtained by formulating ascorbic acid in an oil-in-water emulsion in the presence of a mixture of particular surfactants, a polysaccharide hydrophilic gelling agent and a sulpho-functional polymer.

More specifically the invention provides a composition in oil-in-water emulsion form comprising ascorbic acid or a derivative thereof, at least one polysaccharide hydrophilic gelling agent, at least one C₁₆-C₂₂ fatty acid sorbitan ester, at least one ethoxylated fatty acid ester and at least one polymer containing at least one sulpho-functional monomer.

The invention also provides a non-therapeutic keratin material makeup or care method which comprises applying to the keratin materials the composition defined above.

The composition according to the invention comprises ascorbic acid and/or a derivative thereof, especially its salts or one of its esters. Possible, non-limiting examples include the following: ascorbic acid and its derivatives such as 5,6-di-O-dimethylsilylascorbate (sold by Exsymol under the reference PRO-AA), the potassium salt of dl-alpha-tocopheryl 2-1-ascorbyl phosphate (sold by Senju Pharmaceutical under the reference Sepivital EPC), magnesium ascorbyl phosphate, sodium ascorbyl phosphate (sold by Roche under the reference Stay-C 50) and ascorbyl glucoside (sold by Hayashibara).

The ascorbic acid or derivative thereof may be present in the composition according to the invention in any amount, including preferably an amount ranging from 0.01% to 20% by weight relative to the total weight of the composition, preferably ranging from 0.01% to 10% by weight and more preferably ranging from 0.01% to 5% by weight.

The composition according to the invention comprises at least one polysaccharide hydrophilic gelling agent.

The polysaccharide hydrophilic gelling agent(s) may be selected from glucans, modified and unmodified starches (such as those obtained, for example, from cereals such as wheat, maize or rice, from vegetables such as haricot beans, from root crops such as potatoes or cassava), amylose, amylopectin, glycogen, dextrans, celluloses and derivatives thereof (methylcelluloses, hydroxyalkylcelluloses, hydroxyethylcelluloses, hydroxypropylcellulose, carboxymethylcelluloses), mannans, xylans, lignins, arabans, galactans, galacturonans, chitin, chitosans, glucuronoxylans, arabinoxylans, xyloglucans, glucomannans, pectic acids and pectins, alginic acid and alginates, arabinogalactans, carragheenins, agars, glycosaminoglucans, gums arabic, gums tragacanth, gums ghatti, karaya gums, carob gums, galactomannans such as guar gums and their nonionic derivatives (hydroxypropylguar) and xanthan gums, and mixtures thereof.

Generally speaking, the compounds of this type that can be used in the present invention are selected from those described in particular in Encyclopedia of Chemical Technology, Kirk-Othmer, third edition, 1982, volume 3, pp. 896-900 and volume 15, pp. 439-58, in Polymers in Nature by E. A. MacGregor and C. T. Greenwood, John Wiley & Sons, chapter 6, pp. 240-328, 1980, and in Industrial Gums—polysaccharides and their derivatives, edited by Roy L. Whistler, second edition, Academic Press Inc., the content of these three works being included in their entirety in the present patent application by way of reference.

Preference is given to using xanthan gums.

The polysaccharide hydrophilic gelling agent may be present in the composition according to the invention in any amount, including preferably an amount ranging from 0.01% to 10% by weight relative to the total weight of the composition, preferably ranging from 0.1% to 5% by weight and more preferably ranging from 0.1% to 3% by weight.

The composition according to the invention comprises as emulsifier at least one C₁₆-C₂₂ fatty acid sorbitan ester and at least one ethoxylated fatty acid ester.

The C₁₆-C₂₂ fatty acid sorbitan esters can be formed by esterifying at least one fatty acid containing at least one saturated or unsaturated linear alkyl chain having respectively 16 to 22 carbon atoms with sorbitol. These esters may in particular be selected from sorbitan stearates, behenates, arachidates, palmitates and oleates, and mixtures thereof. Preference is given to using sorbitan stearates and palmitates, more preferably sorbitan stearates.

The C₁₆-C₂₂ fatty acid sorbitan ester present in the composition according to the invention is advantageously solid at a temperature less than or equal to 45° C.

Examples of sorbitan ester useful in the composition according to the invention may include sorbitan monostearate (CTFA name: Sorbitan stearate), sold by Uniqema under the name Span 60, sorbitan tristearate, sold by Uniqema under the name Span 65 V, sorbitan monopalmitate (CTFA name: Sorbitan palmitate), sold by Uniqema under the name Span 40, sorbitan monooleate, sold by Uniqema under the name Span 80 V, and sorbitan trioleate, sold by Uniqema under the name Span 85 V.

The C₁₆-C₂₂ fatty acid sorbitan ester may be present in the composition according to the invention in any amount, including preferably in an amount ranging from 0.01% to 10% by weight relative to the total weight of the composition and preferably ranging from 0.01% to 5% by weight.

The ethoxylated fatty ester present in the composition according to the invention is preferably a C₁₆-C₂₂ fatty acid ester containing 8 to 100 ethylene oxide units.

The fatty chain of the esters may be selected in particular from stearyl, behenyl, arachidyl, palmityl and cetyl units and mixtures thereof such as cetearyl.

The number of ethylene oxide units may range from 8 to 100, preferably from 10 to 80 and more preferably from 20 to 60. In one particular embodiment of the invention this number is 40.

Examples of ethoxylated fatty esters include stearic esters containing respectively 20, 30, 40, 50 or 100 ethylene oxide units, such as the products sold respectively under the names Myrj 49 P, Myrj 51, Myrj 52 P (polyethylene glycol 40 EO stearate; CTFA name: PEG-40 stearate), Myrj 53 and Myrj 59 P by Uniqema.

The ethoxylated fatty ester may be present in the composition according to the invention in any amount, including preferably in an amount ranging from 0.01% to 10% by weight relative to the total weight of the composition and preferably ranging from 0.01% to 5% by weight.

The composition according to the invention further comprises at least one polymer containing at least one sulpho-functional monomer. The presence of this polymer is believed to endow the resulting composition with good cosmetic properties—in particular, absence of stringing, particularly when the composition is taken up with the finger, absence of sticking, and softness—and also promotes effective penetration of the composition when applied to the skin.

The polymers containing at least one sulpho-functional monomer that are used in the composition of the invention are water-soluble or water-dispersible or swellable in water. The polymers used in accordance with the invention may be homopolymers or copolymers and are obtainable from at least one ethylenically unsaturated, sulpho-functional monomer which may be in free form or partially or totally neutralized form. These polymers may optionally contain at least one hydrophobic group and in that case may constitute an amphiphilic polymer (or hydrophobic modified polymer).

More preferably the polymers in accordance with the invention may be partially or totally neutralized by an inorganic base (aqueous sodium hydroxide, potassium hydroxide or ammonia) or an organic base such as mono-, di- or triethanolamine, an aminomethylpropanediol, N-methylglucamine, basic amino acids such as arginine and lysine, and mixtures of these compounds. They are generally neutralized. Neutralized polymers for the purposes of the present invention are polymers which have been totally, or almost totally, neutralized, in other words at least 90% neutralized.

The polymers used in the composition of the invention generally have a number-average molecular weight ranging from 1000 to 20 000 000 g/mol, preferably ranging from 20 000 to 5 000 000 and more preferably from 100 000 to 1 500 000 g/mol.

These polymers according to the invention may be crosslinked or non-crosslinked.

The sulpho-functional monomers of the polymer used in the composition of the invention are selected in particular from vinylsulphonic acid, styrenesulphonic acid, (meth) acrylamido (C₁-C₂₂) alkylsulphonic acids, N-(C₁-C₂₂) alkyl (meth) acrylamido (C₁-C₂₂) alkylsulphonic acids such as undecylacrylamidomethanesulphonic acid, and their partially or totally neutralized forms, and mixtures thereof.

According to one preferred embodiment of the invention the sulpho-functional monomers are selected from (meth)acrylamido(C₁-C₂₂)alkylsulphonic acids, such as, for example, acrylamidomethanesulphonic acid, acrylamidoethanesulphonic acid, acrylamidopropanesulphonic acid, 2-acrylamido-2-methylpropanesulphonic acid, 2-methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid and 2-acrylamido-2,6-dimethyl-3-heptanesulphonic acid, their partially or totally neutralized forms, and mixtures thereof.

More particularly use is made of 2-acrylamido-2-methylpropanesulphonic acid (AMPS) and also its partially or totally neutralized forms.

When the polymers are crosslinked the crosslinking agents may be selected from polyolefinically unsaturated compounds commonly used for crosslinking polymers obtained by free-radical polymerization.

Examples of possible crosslinking agents include divinylbenzene, diallyl ether, dipropylene glycol diallyl ether, polyglycol diallyl ethers, triethylene glycol divinyl ether, hydroquinone diallyl ether, ethylene glycol di(meth)acrylate or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols from the sugar series, or other allyl or vinyl ethers of polyfunctional alcohols, and also allyl esters of derivatives of phosphoric and/or vinylphosphonic acid, or mixtures of these compounds.

In one preferred embodiment of the invention the crosslinking agent is selected from methylenebisacrylamide, allyl methacrylate and trimethylolpropane triacrylate (TMPTA). The degree of crosslinking is in general from 0.01 to 10 mol % and more particularly from 0.2 to 2 mol % relative to the polymer.

When the polymers used are homopolymers they contain only sulpho-functional monomers and, if they are crosslinked, one or more crosslinking agents.

These homopolymers are generally crosslinked and neutralized, and they may be obtained by the preparation process which comprises the following steps:

(a) the monomer, such as 2-acrylamido-2-methylpropanesulphonic acid in free form, is dispersed or dissolved in a solution of tert-butanol or of water and tert-butanol;

(b) the monomer solution or dispersion obtained in (a) is neutralized with one or more organic or inorganic bases, preferably aqueous ammonia, NH₃, in an amount sufficient to give a degree of neutralization of the polymer's sulphonic acid functions of from 90% to 100%;

(c) the solution or dispersion obtained in (b) is admixed with the crosslinking monomer or monomers;

(d) a conventional free-radical polymerization is carried out in the presence of free-radical initiators at a temperature ranging from 10 to 150° C., the polymer precipitating in the solution or dispersion based on tert-butanol.

The preferred AMPS homopolymers are generally wherein they comprise in random distribution:

a) from 90% to 99.9% by weight of units of general formula (II) below: 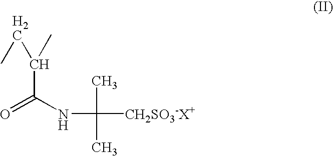
in which X⁺ denotes a proton, an alkali metal cation, an alkaline earth metal cation or the ammonium ion, not more than 10 mol % of the cations X⁺ being protons H⁺;

b) from 0.01% to 10% by weight of crosslinking units originating from at least one monomer having at least two olefinic double bonds, the proportions by weight being defined relative to the total weight of the polymer.

The homopolymers according to the invention that are more particularly preferred contain from 98% to 99.5% by weight of units of formula (II) and from 0.2% to 2% by weight of crosslinking units.

Polymers of this type include, in particular, the crosslinked and neutralized homopolymer of 2-acrylamido-2-methylpropanesulphonic acid, sold by Clariant under the trade name Hostacerin AMPS (CTFA name: Ammonium polyacryldimethyltauramide).

The polymer may also be an amphiphilic homopolymer (or hydrophobic modified homopolymer) selected from random amphiphilic AMPS polymers modified by reaction with a C₆-C₂₂ n-monoalkylamine or di-n-alkylamine, such as those described in document WO-A-00/31154, which are graft homopolymers.

When the polymers used are copolymers they are obtainable from ethylenically unsaturated, sulpho-functional monomers and other ethylenically unsaturated monomers, i.e. ethylenically unsaturated monomers without a sulpho group.

The ethylenically unsaturated, sulpho-functional monomers are selected from those described above.

The ethylenically unsaturated monomers without a sulpho group may be selected from ethylenically unsaturated hydrophilic monomers, ethylenically unsaturated hydrophobic monomers and mixtures thereof. When the polymer contains hydrophobic monomers it constitutes an amphiphilic polymer (also called hydrophobic modified polymer).

The ethylenically unsaturated hydrophilic monomers may be selected for example from (meth)acrylic acids, their β-substituted alkyl derivatives or their esters obtained with monoalcohols or mono- or polyalkylene glycols, or from (meth)acrylamides, vinylpyrrolidone, vinylformamide, maleic anhydride, itaconic acid, maleic acid or mixtures of these compounds.

When the polymer of the composition according to the invention is a copolymer obtainable from ethylenically unsaturated, sulpho-functional monomers and ethylenically unsaturated hydrophilic monomers, it may be selected in particular from (1) crosslinked anionic copolymers of acrylamide or methacrylamide and 2-acrylamido-2-methylpropanesulphonic acid, especially those in the form of an O/W emulsion, such as those sold under the name Sepigel 305 by Seppic (CTFA name: Polyacrylamide/C13-14 isoparaffin/laureth-7), under the name Simulgel 600 by Seppic (CTFA name: Acrylamide/Sodium acryloyldimethyltaurate copolymer/isohexadecane/polysorbate 80), (2) copolymers of (meth)acrylic acid or (meth)acrylate and 2-acrylamido-2-methylpropanesulphonic acid, especially those in the form of a W/O emulsion, such as those sold under the name Simulgel NS by Seppic (sodium acrylamido-2-methylpropanesulphonate/hydroxyethyl acrylate copolymer in inverse emulsion at 40% in polysorbate 60 and squalane) (CTFA name: Hydroxyethyl acrylate/sodium acryloyldimethyltaurate copolymer/squalane/polysorbate 60) or those sold under the name Simulgel EG by Seppic (acrylic/acrylamido-2-methylpropanesulphonic acid copolymer in sodium salt form in inverse emulsion at 45% in isohexadecane/water) (CTFA name: Sodium Acrylate/Sodium acryloyldimethyltaurate copolymer/Isohexadecane/Polysorbate 80), and (3) copolymers of 2-acrylamido-2-methylpropanesulphonic acid and vinylpyrrolidone or vinylformamide, such as the products sold under the name Aristoflex AVC by Clariant.

When the sulpho-functional monomers are copolymerized with ethylenically unsaturated hydrophobic monomers containing a hydrophobic chain, which is also called a fatty chain (C6-C50) chain, the resulting polymer is amphiphilic, which is to say that it contains both a hydrophilic moiety and a hydrophobic moiety. Polymers of this kind are also referred to as hydrophobic modified polymers.

These hydrophobic modified polymers may additionally contain one or more monomers containing neither a sulpho group nor a fatty chain, such as (meth)acrylic acids, their β-substituted alkyl derivatives or their esters obtained with monoalcohols or mono- or polyalkylene glycols, or (meth) acrylamides, vinylpyrrolidone, vinylformamide, maleic anhydride, itaconic acid, maleic acid or mixtures of these compounds.

Hydrophobic modified polymers which can be used include in particular those obtainable from 2-acrylamido-2-methylpropanesulphonic acid (AMPS) and at least one ethylenically unsaturated hydrophobic monomer containing at least one group having 6 to 50 carbon atoms, more preferably 6 to 22 carbon atoms, very preferably 6 to 18 carbon atoms and especially 12 to 18 carbon atoms.

These polymers are described in particular in documents EP-A-750899, U.S. Pat. No. 5,089,578 and WO-A-2002/43689, and in the following publications by Yotaro Morishima:

“Self-assembling amphiphilic polyelectrolytes and their nanostructures”, Chinese Journal of Polymer Science Vol. 18, No. 40 (2000), 323-36;

“Micelle formation of random copolymers of sodium 2-(acrylamido)-2-methylpropanesulfonate and a nonionic surfactant macromonomer in water as studied by fluorescence and dynamic light scattering”, Macromolecules 2000, Vol. 33, No. 10, 3694-704;

“Solution properties of micelle networks formed by nonionic surfactant moieties covalently bound to a polyelectrolyte: salt effects on rheological behavior”, Langmuir, 2000, Vol. 16, No. 12, 5324-332;

“Stimuli responsive amphiphilic copolymers of sodium 2-(acrylamido)-2-methylpropanesulphonate and associative macromonomers”, Polym. Preprints, Div. Polym. Chem. 1999, 40(2), 220-1.

The hydrophobic monomers of these particular polymers are selected preferably from acrylates, alkylacrylates, acrylamides or alkylacrylamides of formula (III) below: 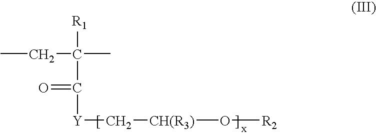
in which R₁ and R₃, which are identical or different, denote a hydrogen atom or a C₁-C₆ branched or substantially linear alkyl radical (preferably methyl), Y denotes O or NH, R₂ denotes a hydrocarbon radical containing 6 to 50 carbon atoms, more preferably 6 to 22 carbon atoms, very preferably 6 to 18 carbon atoms and more particularly 12 to 18 carbon atoms, and x denotes a-number of moles of alkylene oxide and is from 0 to 100.

The radical R₂ is selected preferably from substantially linear C₆-C₁₈ alkyl radicals (for example n-hexyl, n-octyl, n-decyl, n-hexadecyl, n-dodecyl or lauryl, n-octadecyl or stearyl), branched or cyclic C₆-C₁₈ alkyl radicals (for example cyclododecane (C₁₂) or adamantane (C₁₀); perfluorinated C₆-C₁₈ alkyl radicals (for example the group of formula —(CH₂)₂—(CF₂)₉—(CF₃); the cholesteryl radical (C₂₇) or a cholesterol ester residue such as the group cholesterol oxyhexanoate; and aromatic polycyclic groups such as naphthalene or pyrene. Among these radicals more particular preference is given to substantially linear alkyl radicals and more particularly the n-dodecyl, n-hexadecyl or n-octadecyl radical, and mixtures thereof.

In one particularly preferred embodiment of the invention the monomer of formula (III) contains at least one alkylene oxide unit (x≧1) and preferably a plurality of alkylene oxide units (x≧1), constituting a polyoxyalkylene chain. The polyoxyalkylene chain, preferably, is composed of ethylene oxide units and/or propylene oxide units, and more particularly is composed of ethylene oxide units. The number of oxyalkylene units (or number of moles of alkylene oxide) is in general from 3 to 100, more preferably from 3 to 50 and very preferably from 7 to 25.

Among these polymers mention may be made of the following:

crosslinked and non-crosslinked, neutralized and non-neutralized copolymers containing 15% to 60% by weight of AMPS units and 40% to 85% by weight of (C₈-C₁₆)alkyl(meth)acrylamide units or (C₈-C₁₆)alkyl (meth)acrylate units, relative to the polymer, such as those described in the document EP-A-750 899;

terpolymers containing 10 to 90 mol % of acrylamide units, 0.1 to 10 mol % of AMPS units and 5 to 80 mol % of n-(C₆-C₁₈)alkylacrylamide units, relative to the polymer, such as those described in document U.S. Pat. No. 5,089,578;

non-crosslinked copolymers of partially or totally neutralized AMPS and n-dodecyl, n-hexadecyl or n-octadecyl methacrylate, such as those described in the Morishima articles cited above; and

crosslinked or non-crosslinked copolymers of partially or totally neutralized AMPS and n-dodecylmethacrylamide, such as those described in the Morishima articles cited above.

As hydrophobic modified polymers mention may be made more particularly of the copolymers composed (i) of 2-acrylamido-2-methylpropanesulphonic acid (AMPS) units of formula (II) indicated above, in which X⁺ is a proton, an alkali metal cation, an alkaline earth metal cation or ammonium ion, and (ii) of units of formula (IV) below: 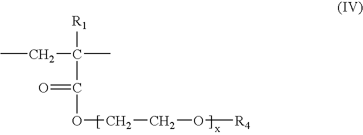
in which x denotes an integer from 3 to 100, preferably from 3 to 50 and more preferably from 7 to 25, R₁ has the same signification as that indicated above in the formula (III) and R₄ denotes a linear or branched alkyl radical containing 6 to 22 carbon atoms and preferably 10 to 22 carbon atoms.

The hydrophobic modified polymers of this type are especially those described in the Morishima articles mentioned above and for which x=25, R₁ denotes methyl and R₄ represents n-dodecyl; or those described in document WO-A-02/43689 and for which x=8 or 25, R₁ denotes methyl and R₄ represents n-hexadecyl (C₁₆), n-octadecyl (C₁₈) or n-dodecyl (C₁₂), or mixtures thereof. The polymers for which X⁺ denotes sodium or ammonium are more particularly preferred.

The preferred hydrophobic modified polymers which can be used in the composition in accordance with the invention may be obtained by conventional processes of free-radical polymerization in the presence of one or more initiators such as, for example, azobisisobutyronitrile (AIBN), azobisdimethylvaleronitrile, 2,2-azobis(2-amidinopropane) hydrochloride (ABAH), organic peroxides such as dilauryl peroxide, benzyl peroxide, tert-butyl hydroperoxide, etc, inorganic peroxide compounds such as potassium persulphate or ammonium persulphate, or H₂O₂, optionally in the presence of reductants.

These hydrophobic modified polymers may be obtained in particular by free-radical polymerization in a tert-butanol medium, in which they precipitate. Using polymerization by precipitation in tert-butanol it is possible to obtain a polymer particle size distribution which is particularly favourable for the uses of the polymer.

The reaction may be conducted at a temperature between 0 and 150° C., preferably between 10 and 100° C., either at atmospheric pressure or under reduced pressure. It may also be carried out under an inert atmosphere, and preferably under nitrogen.

These preferred hydrophobic modified polymers are in particular those described in document EP 1 069 142, and especially those obtained by polymerizing 2-acrylamido-2-methylpropanesulphonic acid (AMPS) or one of its sodium or ammonium salts with an ester of (meth)acrylic acid and of

a C₁₀-C₁₈ alcohol ethoxylated with 8 moles of ethylene oxide (Genapol® C-080 from Clariant),

a C₁₁ oxo alcohol ethoxylated with 8 moles of ethylene oxide (Genapol® UD-080 from Clariant),

a C₁₁ oxo alcohol ethoxylated with 7 moles of ethylene oxide (Genapol® UD-070 from Clariant),

a C₁₂-C₁₄ alcohol ethoxylated with 7 moles of ethylene oxide (Genapol® LA-070 from Clariant),

a C₁₂-C₁₄ alcohol ethoxylated with 9 moles of ethylene oxide (Genapol® LA-090 from Clariant),

a C₁₂-C₁₄ alcohol ethoxylated with 11 moles of ethylene oxide (Genapol® LA-110 from Clariant),

a C₁₆-C₁₈ alcohol ethoxylated with 8 moles of ethylene oxide (Genapol® T-080 from Clariant),

a C₁₆-C₁₈ alcohol ethoxylated with 15 moles of ethylene oxide (Genapol® T-150 from Clariant),

a C₁₆-C₁₈ alcohol ethoxylated with 11 moles of ethylene oxide (Genapol® T-110 from Clariant),

a C₁₆-C₁₈ alcohol ethoxylated with 20 moles of ethylene oxide (Genapol® T-200 from Clariant),

a C₁₆-C₁₈ alcohol ethoxylated with 25 moles of ethylene oxide (Genapol® T-250 from Clariant),

a C₁₈-C₂₂ alcohol ethoxylated with 25 moles of ethylene oxide and/or an iso-C₁₆-C₁₈ alcohol ethoxylated with 25 moles of ethylene oxide.

The molar % concentration of the units of formula (II) and of the units of formula (IV) in the polymers according to the invention varies depending on the desired cosmetic application and the required Theological properties of the formulation. It may be between 0.1 and 99.9 mol %.

Preferably, for the most hydrophobic polymers, the molar proportion of units of formula (II) or (IV) is from 50.1% to 99.9%, more particularly from 70% to 95% and very particularly from 80% to 90%.

Preferably, for the least hydrophobic polymers, the molar proportion of units of formula (II) or (IV) is from 0.1% to 50%, more particularly from 5% to 25% and very particularly from 10% to 20%.

The distribution of the monomers in the polymers of the invention may for example be alternating, blockwise (including multiblock arrangements) or any other.

Hydrophobic modified polymers of this type include especially the copolymer of AMPS and ethoxylated C₁₂-C₁₄ alcohol methacrylate (non-crosslinked copolymer obtained from Genapol LA-070 and AMPS) (CTFA name: Ammonium Acryloyldimethyltaurate/Laureth-7 Methacrylate Copolymer), sold under the name Aristoflex LNC by Clariant, and the copolymer of AMPS and ethoxylated (25 EO) stearyl methacrylate (copolymer crosslinked with trimethylolpropane triacrylate, obtained from Genapol T-250 and AMPS) (CTFA name: Ammonium Acryloyldimethyltaurate/Steareth-25 methacrylate crosspolymer), sold under the name Aristoflex HMS by Clariant.

The polymer containing at least one sulpho-functional monomer that is used in the composition in accordance with the invention is present in an amount of active substance ranging for example from 0.01% to 20% by weight, preferably from 0.1% to 10% by weight, more preferably from 0.1% to 5% by weight and very preferably from 0.5% to 3% by weight relative to the total weight of the composition.

The composition according to the invention comprises an aqueous phase.

The composition may comprise water in any amount, including preferably in an amount ranging from 20% to 95% by weight relative to the total weight of the composition, preferably ranging from 30% to 90% by weight and more preferably ranging from 40% to 70% by weight.

The water may be a floral water such as cornflower water and/or a mineral water such as Vittel water, Lucas water or La Roche Posay water, and/or a thermal water.

The composition may further comprise an organic solvent which is miscible with water at ambient temperature (25° C.) and is selected in particular from monoalcohols having 2 to 6 carbon atoms, such as ethanol and isopropanol;

polyols having in particular 2 to 20 carbon atoms, preferably having 2 to 10 carbon atoms and more preferably having 2 to 6 carbon atoms, such as glycerol, propylene glycol, butylene glycol, pentylene glycol, hexylene glycol, dipropylene glycol and diethylene glycol;

glycol ethers (having in particular 3 to 16 carbon atoms (such as (C₁-C₄)alkyl ethers of mono-, di- or tripropylene glycol and (C₁-C₄)alkyl ethers of mono-, di- or triethylene glycol;

and mixtures thereof.

The composition according to the invention may comprise an organic solvent which is miscible with water at ambient temperature, in particular a polyol, in any amount, including preferably in an amount ranging from 1% to 20% by weight relative to the total weight of the composition and preferably ranging from 3% to 15% by weight.

Advantageously the composition according to the invention has a pH ranging from 3.0 to 8.0, preferably ranging from 4.0 to 8.0, more preferably ranging from 5.0 to 7.0 and very preferably ranging from 5.5 to 6.5.

The emulsion according to the invention further comprises an oily phase.

Oils which can be used more particularly in the composition of the invention include for example:

hydrocarbon oils of animal origin, such as perhydrosqualene (or squalane);

synthetic esters and ethers, particularly of fatty acids, such as the oils of formulae R¹COOR² and R¹OR² in which R¹ represents the residue of a fatty acid containing 8 to 29 carbon atoms and R² represents a branched or unbranched hydrocarbon chain containing 3 to 30 carbon atoms, such as, for example, Purcellin oil, isononyl isononanoate, isopropyl myristate, 2-ethylhexyl palmitate, 2-octyldodecyl stearate, 2-octyldodecyl erucate, and isostearyl isostearate; hydroxy esters such as isostearyl lactate, octyl hydroxystearate, octyldodecyl hydroxystearate, diisostearyl malate and triisocetyl citrate, and fatty alcohol heptanoates, octanoates and decanoates; polyol. esters, such as propylene glycol dioctanoate, neopentyl glycol diheptanoate and diethylene glycol diisononanoate; pentaerythritol esters such as pentaerythrityl tetraisostearate; and lipophilic derivatives of amino acids, such as isopropyl lauroylsarcosinate (INCI name: Isopropyl Lauroylsarcosinate), sold under the name Eldew SL 205 by Ajinomoto;

linear or branched hydrocarbons of mineral or synthetic origin, such as mineral oils (mixture of petroleum-derived hydrocarbon oils; INCI name: Mineral oil), volatile or non-volatile liquid paraffins and derivatives thereof, petroleum jelly, polydecenes, isohexadecane, isododecane, hydrogenated isoparaffin such as the Parleam® oil sold by NOF Corporation (INCI name: Hydrogenated Polyisobutene);

silicone oils such as volatile or non-volatile polydimethylsiloxanes (PDMS) containing a linear or cyclic silicone chain which are liquid or pasty at ambient temperature, especially cyclopolydimethylsiloxanes (cyclomethicones) such as cyclopentadimethylsiloxane and cyclohexadimethyl-siloxane; polydimethylsiloxanes containing alkyl, alkoxy or phenyl groups which are pendent or at the end of the silicone chain, these groups having 2 to 24 carbon atoms; phenyl silicones such as phenyltrimethicones, phenyldimethicones, phenyltrimethylsiloxydiphenylsiloxanes, diphenyldimethicones, diphenylmethyldiphenyltrisiloxanes, 2-phenylethyl trimethylsiloxysilicates and polymethylphenylsiloxanes;

fluoro oils, such as those which are partially hydrocarbon-based and/or silicone-based, such as those described in document JP-A-2-295912;

ethers such as dicaprylyl ether (CTFA name: Dicaprylyl ether); and C₁₂-C₁₅ fatty alcohol benzoates (Finsolv TN from Finetex); and

mixtures thereof.

The oil may be present in the composition according to the invention in any amount, including preferably in an amount ranging from 1% to 50% by weight relative to the total weight of the composition, preferably ranging from 5% to 40% by weight and more preferably ranging from 5% to 30% by weight.

The oily phase of the emulsion may comprise other fatty substances such as waxes, gums such as silicone gums (dimethiconol), silicone resins, and mixtures thereof.

The composition according to the invention may comprise at least one organic photoprotectant which is active in UVA and/or UVB (absorbers), these photoprotectants being water-soluble or fat-soluble or else insoluble in commonly used cosmetic solvents.

Being of good stability, the composition according to the invention is appropriate for the formulation of organic UV screens; the UV screens incorporated in the composition are not degraded in the presence of vitamin C.

The organic screens are selected in particular from anthranilates; cinnamic derivatives; dibenzoylmethane derivatives; salicylic derivatives; camphor derivatives; triazine derivatives other than those of the invention, such as those described in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243, EP 944 624; benzophenone derivatives; β,β-diphenylacrylate derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives, as described in applications U.S. Pat. No. 5,237,071, U.S. Pat. No. 5,166,355, GB 2303549, DE 197 26 184 and EP 893 119; polymer screens and silicone screens, such as those described in particular in application WO 93/04665; dimers derived from α-alkylstyrene, such as those described in patent application DE 198 55 649; 4,4-diarylbutadienes as described in applications EP 0967200, DE 197 46 654, DE 197 55 649, EP-A-1 008 586, EP 1 133 980 and EP 1 133 981, and mixtures thereof.

As examples of organic screens mention may be made of those denoted below by their INCI name:

Para-Aminobenzoic Acid Derivatives:

PABA,
Ethyl PABA,
Ethyl Dihydroxypropyl PABA,
Ethylhexyl Dimethyl PABA, sold in particular under the name Escalol 507 by ISP,
Glyceryl PABA,
PEG-25 PABA, sold under the name Uvinul P25 by BASF.
   Salicylic Derivatives:
Homosalate, sold under the name Eusolex HMS by Rona/EM Industries,
Ethylhexyl Salicylate, sold under the name Neo Heliopan OS by Haarmann & Reimer,
Dipropylene glycol salicylate, sold under the name Dipsal by Scher,
TEA Salicylate, sold under the name Neo Heliopan TS by Haarmann & Reimer.
   Dibenzoylmethane Derivatives:
Butyl methoxydibenzoylmethane, sold in particular under the trade name Parsol 1789 by Hoffmann La Roche,
Isopropyl Dibenzoylmethane.
   Cinnamic Derivatives:
Ethylhexyl methoxycinnamate, sold in particular under the trade name Parsol MCX by Hoffmann La Roche,
Isopropyl Methoxycinnamate,
Isoamyl Methoxycinnamate, sold under the trade name Neo Heliopan E 1000 by Haarmann & Reimer,
DEA Methoxycinnamate,
Diisopropyl Methylcinnamate,
Glyceryl Ethylhexanoate Dimethoxycinnamate.
   β,β-Diphenylacrylate Derivatives:
Octocrylene, sold in particular under the trade name Uvinul N539 by BASF,
Etocrylene, sold in particular under the trade name Uvinul N35 by BASF.
   Benzophenone Derivatives:
Benzophenone-1, sold under the trade name Uvinul 400 by BASF,
Benzophenone-2, sold under the trade name Uvinul D50 by BASF,
Benzophenone-3 or oxybenzone, sold under the trade name Uvinul M40 by BASF,
Benzophenone-4, sold under the trade name Uvinul MS40 by BASF,
Benzophenone-5,
Benzophenone-6, sold under the trade name Helisorb 11 by Norquay,
Benzophenone-8, sold under the trade name Spectra-Sorb UV-24 by American Cyanamid,
Benzophenone-9, sold under the trade name Uvinul DS-49 by BASF,
Benzophenone-12
Diethylamino Hydroxybenzoyl Hexyl Benzoate, sold under the trade name Uvinul A Plus by BASF.
   Benzylidenecamphor Derivatives:
3-Benzylidene camphor, manufactured under the name Mexoryl SD by Chimex,
4-Methylbenzylidene camphor, sold under the name Eusolex 6300 by Merck,
Benzylidene Camphor Sulphonic Acid, manufactured under the name Mexoryl SL by Chimex,
Camphor Benzalkonium Methosulphate, manufactured under the name Mexoryl SO by Chimex,
Terephthalylidene Dicamphor Sulphonic Acid, manufactured under the name Mexoryl SX by Chimex,
Polyacrylamidomethyl Benzylidene Camphor, manufactured under the name Mexoryl SW by Chimex.
   Phenylbenzimidazole Derivatives:
Phenylbenzimidazole Sulphonic Acid, sold in particular under the trade name Eusolex 232 by Merck,
Disodium Phenyl Dibenzimidazole Tetra-sulphonate, sold under the trade name Neo Heliopan AP by Haarmann & Reimer.
   Triazine Derivatives:
Bis-Ethylhexyloxyphenol Methoxyphenyl Triazine, sold under the trade name Tinosorb S by Ciba Geigy,
Ethylhexyl triazone, sold in particular under the trade name Uvinul T150 by BASF,
Diethylhexyl Butamido Triazone, sold under the trade name Uvasorb Heb by Sigma 3V.
   Phenylbenzotriazole Derivatives:
Drometrizole Trisiloxane, sold under the name Silatrizole by Rhodia Chimie,
Methylene bis-Benzotriazolyl Tetramethylbutylphenol, sold in solid form under the trade name Mixxim BB/100 by Fairmount Chemical or in micronized form in aqueous dispersion under the trade name Tinosorb M by Ciba Specialty Chemicals.
   Anthranilic Derivatives:
Menthyl anthranilate, sold under the trade name Neo Heliopan MA by Haarmann & Reimer.
   Imidazoline Derivatives:
Ethylhexyl Dimethoxybenzylidene Dioxoimidazoline Propionate.
   Benzalmalonate Derivatives:
Polyorganosiloxanes containing a benzalmalonate function, such as Polysilicone-15, sold under the trade name Parsol SLX by Hoffmann La Roche.
   4,4-Diarylbutadiene Derivatives:
1,1-dicarboxy(2,2′-dimethylpropyl)-4,4-diphenyl-butadiene.
   Benzoxazole derivatives:
2,4-bis[5-1-(dimethylpropyl)benzoxazol-2-yl-(4-phenyl)imino]-6-(2-ethylhexyl)imino-1,3,5-triazine, sold under the name Uvasorb K2A by Sigma 3V;
and mixtures thereof.
   The preferred organic UV screens are selected from
Ethylhexyl Salicylate,
Ethylhexyl Methoxycinnamate,
Butyl Methoxydibenzoylmethane,
Octocrylene,
Phenylbenzimidazole Sulphonic Acid,
Disodium Phenyl Dibenzimidazole Tetrasulphonate,
Benzophenone-3,
Benzophenone-4,
Benzophenone-5,
n-hexyl 2-(4-diethylamino-2-hydroxybenzoyl)benzoate,
4-Methylbenzylidene camphor,
Terephthalylidene Dicamphor Sulphonic Acid,
Bis-Ethylhexyloxyphenol Methoxyphenyl Triazine,
Ethylhexyl triazone,
Diethylhexyl Butamido Triazone,
Methylene bis-Benzotriazolyl Tetramethylbutylphenol,
Drometrizole Trisiloxane,
Polysilicone-15,
1,1-dicarboxy(2,2′-dimethylpropyl)-4,4-diphenyl-butadiene,
2,4-bis[5-1-(dimethylpropyl)benzoxazol-2-yl-(4-phenyl)imino]-6-(2-ethylhexyl)imino-1,3,5-triazine
and mixtures thereof.

Advantageously a nonionic organic protective screen is used.

The photoprotectant may be present in the composition according to the invention in an amount ranging from 0.01% to 30% by weight relative to the total weight of the composition, preferably ranging from 0.1% to 25% by weight and more preferably ranging from 0.1% to 20% by weight.

The composition according to the invention may further comprise fillers.

By fillers are meant particles of any shape which are colourless or white, organic or inorganic, which are insoluble in the medium of the composition irrespective of the temperature at which the composition is manufactured, and which do not colour the composition.

The fillers may be of any shape, platelet-like, spherical or oblong, irrespective of the crystallographic form (for example sheet, cubic, hexagonal, orthorhombic, etc). Mention may be made of talc, mica, silica, kaolin, poly-β-alanine and polyethylene, powders of tetrafluoroethylene polymers (Teflon®), lauroyllysine, starch, boron nitride, polymeric hollow microspheres such as those of polyvinylidene chloride/acrylonitrile such as Expancel® (Nobel Industries), copolymers of acrylic acid, silicone resin microbeads (Tospearls® from Toshiba, for example), particles of elastomeric polyorganosiloxanes, precipitated calcium carbonate, magnesium carbonate and hydrogencarbonate, hydroxyapatite, barium sulphate, aluminium oxides, polyurethane powders, composite fillers, hollow silica microspheres, and ceramic or glass microcapsules.

The fillers may be present in the composition in any amount, including preferably in an amount ranging from 0.1% to 15% by weight and preferably ranging from 0.1% to 10% by weight and more preferably from 0.1% to 5% by weight relative to the total weight of the composition.

The composition according to the invention may further comprise an active selected from desquamating agents capable of acting either by promoting exfoliation or on the enzymes involved in desquamation or corneodesmosome degradation, moisturizers, depigmenting or pro-pigmenting agents, anti-glycation agents, NO synthase inhibitors, 5α-reductase inhibitors, lysyl and/or proplyl hydroxylase inhibitors, agents which stimulate the synthesis of dermal or epidermal macromolecules and/or prevent their degradation, agents which stimulate the proliferation of fibroblasts or keratinocytes and/or keratinocyte differentiation, muscle relaxants, antimicrobials, tensing agents, anti-pollutants or free-radical scavengers, antiinflammatories, lipolytic actives or actives having favourable, direct or indirect, activity on the reduction of fatty tissue, agents acting on the microcirculation, and agents acting on cell energy metabolism.

Advantageously the emulsion according to the invention may have a viscosity as measured at 25° C. and a shear rate of 200 min⁻¹ (200 revolutions per minute, or a frequency of 50 Hz) ranging from 1.5 to 2 Pa.s (15 to 20 poises) and preferably ranging from 1.7 to 1.9 Pa.s (17 to 19 poises). The viscosity is measured at 25° C. with a Mettler Rheomat 180 viscometer equipped with a No. 3 spindle, measurement being made after 10 minutes of spindle rotation (after which stabilization of the viscosity and of the rotational speed of the spindle is observed) at a shear rate of 200 min⁻¹.

The composition according to the invention is intended in particular for topical use, especially cosmetic or dermatological topical use.

The composition of the invention may also include adjuvants such as those which are commonplace in cosmetology or dermatology, such as preservatives, perfumes, bactericides, odour absorbers, colorants, salts, surfactants, thickeners and bases. The amounts of these various adjuvants are those conventionally used in the field in question, and amount for example to 0.01% to 20% of the total weight of the composition. These adjuvants, depending on their nature, may be introduced into the fatty phase or into the aqueous phase.

The composition according to the invention may be applied to the skin, body hair, lashes, head hair, nails or lips, depending on the use for which the composition is intended. Hence it may be used in a cosmetic skin treatment method which comprises applying the composition according to the invention to the skin for the purpose, for example, of toning it, regenerating it, smoothing out the skin's fine lines and/or for combating skin ageing, for combating the harmful effects of UV radiation and/or for strengthening the skin tissues against environmental attack.

In one version the composition according to the invention may be used for manufacturing a dermatological preparation, such as a preparation intended for depigmenting the skin, body hair and/or head hair.

The composition may be a care composition and in particular may be a skincare product such as a skincare base, a care cream (day cream, night cream, anti-wrinkle cream) or a makeup base, a lip care composition (lip balm) or a sun protection or self-tanning composition.

The composition may also be a makeup composition, in particular a skin, lip, eyelash, eyebrow or hair makeup composition. The makeup composition may in particular be a foundation, blusher, eye shadow, concealer or body makeup product.

Advantageously the composition is one used without rinsing.

The emulsion according to the invention may be prepared in accordance with the following general procedure:

Mix the constituents of the aqueous phase with heating at a temperature of approximately 70° C. Also mix the oils and surfactants with heating at a temperature of approximately 80° C. Pour the fatty phase into the aqueous phase at a temperature of approximately 70° C. and then stir at high speed, using a turbine for 10 minutes. Cool the resulting emulsion to approximately 60° C. Then add the thickeners, and subsequently stir again for 10 minutes. Cool to approximately 50° C. Thereafter introduce the ascorbic acid or derivative, mixed beforehand with water, and then the other actives where appropriate.

The invention will now be illustrated with the aid of the following, non-limiting examples.

### EXAMPLE 1

A facecare composition was prepared in the form of a water-in-oil emulsion having the following composition:

| Sorbitan tristearate (SPAN 65 V from Uniqema) | 0.9 | g |
|---|---|---|
| PEG (40 EO) stearate (Myrj 52 P from Uniqema) | 2 | g |
| Cetyl alcohol | 2 | g |
| Mixture of glyceryl mono- and distearate (36/64) | 1.5 | g |
| Stearic acid | 1 | g |
| 4-tert-Butyl-4′-methoxydibenzoylmethane | 3 | g |
| 2-Ethylhexyl 2-cyano-3,3-diphenylacrylate | 7 | g |
| 2-Ethylhexyl salicylate | 5 | g |
| Liquid petroleum jelly | 4 | g |
| Silicone oil | 10 | g |
| Ascorbic acid | 3 | g |
| Sodium hydroxide | 0.67 | g |
| Xanthan gum | 0.2 | g |
| Acrylamide/sodium acrylamido-2-methylpropane- | 2 | g |
| sulphonate copolymer in emulsion, containing 40% | | |
| of active substance (Simulgel ® 600 from SEPPIC) | | |
| Filler | 3 | g |
| Anti-wrinkle active | 1 | g |
| Glycerol | 3 | g |
| Preservatives | qs | |
| Water | qs 100 | g |

This emulsion exhibits good stability after 8 days at 55° C. and after 2 months at ambient temperature.

The composition is applied to the face in daily day use.

### EXAMPLE 2—COMPARATIVE

A composition is prepared which is similar to that of Example 1 but contains no xanthan gum (the amount of 0.2 g being replaced by water) (this composition does not form part of the invention).

The resulting composition is unstable after storage for 3 days at 55° C., with surface release of the oily phase.

The above written description of the invention provides a manner and process of making and using it such that any person skilled in this art is enabled to make and use the same, this enablement being provided in particular for the subject matter of the appended claims, which make up a part of the original description and including a composition in oil-in-water emulsion form comprising ascorbic acid or a derivative thereof, at least one polysaccharide hydrophilic gelling agent, at least one C₁₆-C₂₂ fatty acid sorbitan ester, at least one ethoxylated fatty acid ester and at least one polymer containing at least one sulpho-functional monomer.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

As used herein, where a certain polymer is noted as being “obtained from” or “comprising”, etc. one or more monomers (or monomer units) this description is of the finished polymer material itself and the repeating units therein that make up, in whole or part, this finished product. One of ordinary skill in the art understands that, speaking precisely, a polymer does not include individual, unreacted “monomers,” but instead is made up of repeating units derived from reacted monomers.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition in oil-in-water emulsion form comprising ascorbic acid and/or at least one derivative thereof, at least one polysaccharide hydrophilic gelling agent, at least one C₁₆-C₂₂ fatty acid sorbitan ester, at least one ethoxylated fatty acid ester, and at least one polymer comprising at least one sulpho-functional monomer unit.

2. The composition according to claim 1, wherein the ascorbic acid derivatives are selected from 5,6-di-O-dimethylsilylascorbate, the potassium salt of dl-alpha-tocopheryl 2l-ascorbyl phosphate, magnesium ascorbyl phosphate, sodium ascorbyl phosphate and ascorbyl glucoside.

3. The composition according to claim 1, wherein it comprises ascorbic acid.

4. The composition according to claim 1, wherein said ascorbic acid and/or derivative thereof is present in an amount of 0.01% to 20% by weight relative to the total weight of the composition.

5. The composition according to claim 1, wherein the polysaccharide hydrophilic gelling agent is selected from glucans, modified starches, unmodified starches, amylose, amylopectin, glycogen, dextrans, celluloses and derivatives thereof, mannans, xylans, lignins, arabans, galactans, galacturonans, chitin, chitosans, glucuronoxylans, arabinoxylans, xyloglucans, glucomannans, pectic acids and pectins, alginic acid and alginates, arabinogalactans, carragheenins, agars, glycosaminoglucans, gums arabic, gums tragacanth, gums ghatti, karaya gums, carob gums, galactomannans, xanthan gums, and mixtures thereof.

6. The composition according to claim 1, wherein the polysaccharide hydrophilic gelling agent is selected from xanthan gums.

7. The composition according to claim 1, wherein the polysaccharide hydrophilic gelling agent is present in an amount of 0.01% to 10% by weight relative to the total weight of the composition.

8. The composition according to claim 1, wherein the C₁₆-C₂₂ fatty acid sorbitan ester is selected from sorbitan stearates, sorbitan behenates, sorbitan arachidates, sorbitan palmitates, sorbitan oleates and mixtures thereof.

9. The composition according to claim 1, wherein the C₁₆-C₂₂ fatty acid sorbitan ester is selected from sorbitan stearates and sorbitan palmitates.

10. The composition according to claim 1, wherein the C₁₆-C22 fatty acid sorbitan ester is selected from sorbitan stearates.

11. The composition according to claim 1, wherein the C₁₆-C₂₂ fatty acid sorbitan ester is selected from sorbitan monostearate and sorbitan tristearate.

12. The composition according to claim 1, wherein the C₁₆-C₂₂ fatty acid sorbitan ester is present in an amount of 0.01% to 10% by weight relative to the total weight of the composition.

13. The composition according to claim 1, wherein the ethoxylated fatty ester is an ester of C₁₆-C₂₂ fatty acids containing 8 to 100 ethylene oxide units.

14. The composition according to claim 1, wherein the ethoxylated fatty ester is an ester of C₁₆-C₂₂ fatty acids containing 10 to 80 ethylene oxide units.

15. The composition according to claim 1, wherein the ethoxylated fatty ester is an ester of C₁₆-C₂₂ fatty acids containing 40 ethylene oxide units.

16. The composition according to claim 1, comprising polyethylene glycol (40 EO) stearate.

17. The composition according to claim 1, wherein the ethoxylated fatty ester is present in an amount of 0.01% to 10% by weight relative to the total weight of the composition.

18. The composition according to claim 1, wherein the sulpho-functional monomer is selected from vinylsulphonic acid, styrenesulphonic acid, (meth)acrylamido(C₁-C₂₂)alkylsulphonic acids, N-(C₁-C₂₂)alkyl (meth) acrylamido(C1-C₂₂)alkylsulphonic acids, their partially or totally neutralized forms, and mixtures thereof.

19. The composition according to claim 1, comprising 2-acrylamido-2-methylpropanesulphonic acid.

20. The composition according to claim 1, comprising a neutralized, crosslinked homopolymer of 2-acrylamido-2-methylpropanesulphonic acid.

21. The composition according to claim 1, comprising at least one random amphiphilic 2-acrylamido-2-methylpropanesulphonic acid polymer modified by reaction with a C₆-C₂₂ n-monoalkylamine or di-n-alkylamine.

22. The composition according to claim 1, wherein the polymer containing at least one sulpho-functional monomer is selected from crosslinked anionic copolymers of acrylamide or methylacrylamide and 2-acrylamido-2-methylpropanesulphonic acid, copolymers of (meth)acrylic acid or (meth)acrylate and 2-acrylamido-2-methylpropanesulphonic acid and copolymers of 2-acrylamido-2-methylpropanesulphonic acid and vinylpyrrolidone or vinylformamide.

23. The composition according to claim 1, wherein the polymer containing at least one sulpho-functional monomer is a hydrophobic modified polymer obtained from 2-acrylamido-2-methylpropanesulphonic acid and at least one ethylenically unsaturated hydrophobic monomer containing at least one group having 6 to 50 carbon atoms.

24. The composition according to claim 23, wherein the hydrophobic modified polymer is selected from:
crosslinked and non-crosslinked, neutralized and non-neutralized copolymers containing 15% to 60% by weight of AMPS units and 40% to 85% by weight of (C₈-C₁₆)alkyl (meth)acrylamide units or (C₈-C₁₆)alkyl (meth)acrylate units, relative to the polymer;
terpolymers containing 10 to 90 mol % of acrylamide units, 0.1 to 10 mol % of AMPS units and 5 to 80 mol % of n-(C₆-C₁₈)alkylacrylamide units, relative to the polymer;
non-crosslinked copolymers of partially or totally neutralized AMPS and n-dodecyl, n-hexadecyl or n-octadecyl methacrylate; and
crosslinked or non-crosslinked copolymers of partially or totally neutralized AMPS and n-dodecylmethacrylamide.

25. The composition according to claim 23, wherein the hydrophobic modified polymer is selected from copolymers composed (i) of 2-acrylamido-2-methylpropanesulphonic acid units of formula (II): 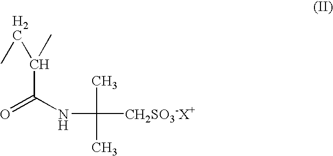
in which X⁺ is a proton, an alkali metal cation, an alkaline earth metal cation or the ammonium ion, and (ii) of units of formula (III) below: 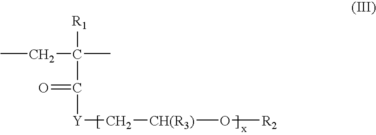
where R₁ and R₃, which are identical or different, denote a hydrogen atom or a C₁-C₆ branched or linear alkyl radical, Y denotes O or NH, R₂ denotes a hydrocarbon radical containing 6 to 50 carbon atoms, and x denotes a number of moles of alkylene oxide and is from 0 to 100.

26. The composition according to claim 1, wherein the polymer containing at least one sulpho-functional monomer is present in an amount of 0.01% to 20% by weight relative to the total weight of the composition.

27. The composition according to claim 1, wherein it comprises water in an amount of 20% to 95% by weight relative to the total weight of the composition.

28. The composition according to claim 1, wherein it further comprises at least one organic solvent which is miscible with water at ambient temperature.

29. The composition according to claim 28, wherein the organic solvent which is miscible with water at ambient temperature is selected from monoalcohols having 2 to 6 carbon atoms, polyols having 2 to 20 carbon atoms, (C₁-C₄)alkyl ethers of mono-, di- or tripropylene glycol, (C₁-C₄)alkyl ethers of mono-, di- or triethylene glycol, and mixtures thereof.

30. The composition according to claim 28, wherein the organic solvent which is miscible with water at ambient temperature is selected from ethanol, isopropanol, glycerol, propylene glycol, butylene glycol, pentylene glycol, hexylene glycol, dipropylene glycol, diethylene glycol and mixtures thereof.

31. The composition according to claim 28, wherein the organic solvent which is miscible with water at ambient temperature is present in an amount of 1% to 20% by weight relative to the total weight of the composition.

32. The composition according to claim 1, wherein it has a pH ranging from 3.0 to 8.0.

33. The composition according to claim 1, wherein it comprises oil in an amount ranging from 1% to 50% by weight relative to the total weight of the composition.

34. The composition according to claim 1, wherein it further comprises at least one organic photoprotectant which is active in UVA and/or UVB.

35. The composition according to claim 34, wherein the photoprotectant is present in an amount ranging from 0.01% to 30% by weight relative to the total weight of the composition.

36. The composition according to claim 1, wherein it has a viscosity, as measured at 25° C. and a shear rate of 200 min⁻¹, ranging from 1.5 to 2 Pa.s.

37. The composition according to claim 1, wherein it is a cosmetic or dermatological composition.

38. The composition according to claim 1, wherein it further comprises at least one cosmetic or dermatological adjuvant selected from fillers, preservatives, perfumes, bactericides, odour absorbers, colorants, salts, surfactants, thickeners and bases.

39. A method which comprises applying to a keratin material the composition according to claim 1.

40. The composition according to claim 39, wherein said composition is applied to a keratin material in order to tone it, regenerate it, smooth out fine lines and/or to combat skin ageing, to combat the harmful effects of UV radiation and/or to strengthen skin tissues against environmental attack.
